(19) United States
(12) Patent Application Publication
(10) Pub. No.: US 2004/0024041 A1
(43) Pub. Date: Feb. 5, 2004

(54) Dermal therapeutic system containing non-steroidal antiphlogistics with selective cox-2 inhibition

(76) Inventor: Torsten Selzer, Neuwied (DE)

Correspondence Address: William F Lawrence, Frommer Lawrence & Haug, 745 Fifth Avenue, New York, NY, 10151, US

(21) Appl. No.: 10/312,417

(22) PCT Filed: Jun. 19, 2001
(86) PCT No.: PCT/EP01/06905

(30) Foreign Application Priority Data
Jul. 1, 2000 (DE) 100 32 132.1

(51) Int. Cl. A61K 31/415; A61K 31/365

(52) U.S. Cl. 514/406; 514/473

(57) ABSTRACT

The invention relates to a dermal therapeutic system which enables a COX-2 inhibitor to be continuously supplied to the skin and the adjacent articular structures over a long period of time. Said dermal therapeutic system can either be a transdermal therapeutic system (TTS) or a superficial therapeutic system (STS).

The invention relates to a dermal therapeutic system which makes it possible to deliver non-steroidal anti-inflammatory drugs with selective COX-2 inhibition to the skin, in particular the human skin.

Dermal therapeutic systems can be divided into transdermal therapeutic systems (TTS) and superficial therapeutic systems (STS).

Transdermal therapeutic systems are dosage forms which are applied to the skin and are designed to make a medicinal substance systemically available. TTS are able to increase the therapeutic value of administration of a medicinal substance by ensuring continuous delivery of the medicinal substance into the blood compartment over a prolonged period. Problems such as gastrointestinal intolerance, low enteral absorption, first pass metabolism in the liver and increased frequency of administration in the case of low half-lives can thus be circumvented.

Superficial therapeutic systems (STS) are dosage forms which are applied to the skin and are designed to make a medicinal substance topically available for the skin. STS are able to increase the therapeutic value of topical administration of a medicinal substance by ensuring continuous delivery of the medicinal substance into the skin compartment over a prolonged period.

Dermal therapeutic systems consist according to the prior art of a support layer which is impermeable to medicinal substance, of a reservoir or matrix layer containing medicinal substance, where appropriate of a control membrane, and of a contact adhesive layer for attachment to the skin, it being possible for the latter to be identical to the layer containing medicinal substance, and of a protective layer which is to be removed before application and is likewise impermeable to medicinal substance. The layer containing medicinal substance consists of medicinal substance and excipients such as, for example, plasticizers, tackifiers, solubilizers, stabilizers, fillers, carriers and permeation promoters. The pharmaceutically acceptable substances suitable for these purposes are known to the skilled person.

Non-steroidal anti-inflammatory drugs (NSAID) such as, for example, diclofenac have been known for some time. They are frequently administered orally, parenterally or else as ointments for the treatment of rheumatoid diseases, inflammations of joints, arthritic and degenerative joint disorders and for pain associated with these disorders. However, dangerous side effects which may occur especially on systemic administration are, inter alia, gastrointestinal bleeds and perforations in the gastrointestinal system. A new category of non-steroidal compounds with anti-inflammatory activity which selectively inhibit the enzyme cyclooxygenase 2 and have fewer or weaker side effects than the abovementioned NSAIDs has since been developed. In particular there is a marked reduction in the risk of the occurrence of gastrointestinal complications. These compounds, which are called COX-2 inhibitors, include, for example, diaryl-2(5H)-furanones and their analogs, in particular 3-aryl-4-(4-methylsulfonylphenyl)-2-furanones (Chan, C. -C., et al., J. Pharmacol. Exp. Ther. 290 (1999), 551-560), and compounds of the 4-[5-aryl-3-(trifluoromethyl)pyrazol-1-yl]benzenesulfonamide class (Scarpignato, C. et al., Gastroenterol. Int. 12/4 (1999), 186-215; Bjarnason, I., Ital. J. Gastroenterol. Hepatol. 31, Suppl. 1 (1999), 27-36; WO 95/15316).

Rofecoxib (3-phenyl-4-[4-methylsulfonylphenyl]-2-(5H)furanone) is the first non-steroidal anti-inflammatory drug with selective COX-2 inhibition which is available on the market. Rofecoxib has been investigated in several clinical studies for the symptomatic treatment of arthritic irritation. In the clinical studies, perforations, ulcers and bleeds in the upper gastrointestinal tract were significantly fewer in patients receiving rofecoxib therapy than in patients receiving therapy with other non-steroidal anti-inflammatory drugs which served as comparison substance. In two endoscopy studies lasting 24 weeks on patients there was a comparable percentage of patients with endoscopically detectable gastroduodenal ulcers after 12 weeks of treatment with placebo and with rofecoxib 25 or 50 mg/day. In both studies the cumulative incidence of gastroduodenal ulcers over a period of 12 and 24 weeks, respectively, was significantly less with rofecoxib than with ibuprofen 2 400 mg/day.

Rofecoxib is particularly indicated for the treatment of symptoms associated with irritation from degenerative joint disorders (arthroses). In the USA, the substance is also approved as an anti-inflammatory agent.

However, systemic administration of rofecoxib and other COX-2 inhibitors is likewise associated with unwanted side effects (although to a lesser extent). Examples which should be mentioned are edemas, accumulations of fluid, abdominal pains, drowsiness, hypertension, heartburn, nausea, vomiting, diarrhea, CNS disturbances such as headaches, dizziness, vomiting, tinnitus, visual disturbances or somnolence. Serious side effects such as nephrotoxicity, hepatotoxicity and gastrointestinal toxicity cannot be precluded on prolonged treatment with COX-2 inhibitors.

It is therefore an object of the present invention to provide a dosage form for the COX-2 inhibitor, in particular rofecoxib, which provides the active ingredient as selectively as possible to the target organ which is to be treated and is affected by arthrosis, inflammations or other rheumatic disorders.

This object is achieved according to the invention by a dermal therapeutic system which is designed to ensure a continuous delivery of the active ingredient (COX-2 inhibitor) to the skin and the surrounding articular structures over a prolonged period.

The systems of the invention can be employed both in the form of matrix systems and in the form of membrane or reservoir systems. It is immaterial which polymers, resins and possibly other additives are employed as long as the formulation is suitable for delivering the active ingredient COX-2 inhibitor to the skin.

In the simplest case, the active ingredient may be in the form of a coarse, colloidal or molecular dispersion in a solution of basic polymers, and possibly the mixture can be coated onto a suitable substrate—ordinarily a siliconized thermoplastic sheet—and, after evaporation of the solvent contents, be covered with another sheet which represents the subsequent back of the TTS/STS. TTS/STS are obtained from such a laminate by punching out sheet-like structures in the desired geometric shape.

Suitable base materials for the system of the invention are polymers based on acrylic acid or methacrylic acid and esters thereof, isobutylene, ethylene/vinyl acetate, elastomers, styrene/diene copolymers, synthetic elastomers or hotmelt adhesive. This list is far from complete but does indicate the wide applicability of the principle of the invention.

The TTS/STS of the invention may additionally include two or more matrix layers, it being possible for the individual matrix layers to contain different concentrations of active ingredient. The individual matrix layers may moreover have different contact adhesives. In order to make it possible to control the release, unless this is brought about by other mechanisms, the reservoir may also be provided with a control membrane which controls delivery of the active ingredient to the skin.

The structure of the TTS/STS of the invention additionally comprises a backing layer which is impermeable to active ingredient, and a protective sheet or detachable sheet which is likewise impermeable to active ingredient. Suitable as backing layer are, in particular, polyesters which are distinguished by particular strength, but also virtually any other skin-compatible synthetics such as, for example, polyvinyl chloride, ethylene/vinyl acetate, vinyl acetate, polyethylene, polypropylene, cellulose derivatives and many others. The backing layer may in the individual case be provided with an additional layer, e.g. by vapor deposition of metals or other diffusion-blocking additives such as silica, alumina or similar substances known to the skilled person. The same materials can be used for the detachable protective layer as for the backing layer, provided that they are detachable through a suitable surface treatment such as, for example, siliconization. However, it is also possible to use other detachable protective layers such as polytetrafluoroethylene-treated paper, cellophane, polyvinyl chloride or the like.

In a particular embodiment, however, the active ingredient may also be present in a bag-like reservoir which is filled with a high viscosity liquid, semisolid or thixotropic matrix which contains the active ingredient. It is particularly advantageous if the semisolid or thixotropic active ingredient reservoir contains a gel former. The reverse side of the bag facing away from the skin must in this case be impermeable to active ingredient. It is optionally possible for a membrane which is permeable to active ingredient to take over control of the release of active ingredient.

Suitable excipients present in one or more matrix layers are, for example, plasticizers. These may be, depending on the polymer used: higher alcohols such as dodecanal, undecanol, octanol, carboxylic esters such as n-butyl adipate, triglcerides, or polyhydric alcohols.

If a transdermal therapeutic system (TTS) is used for administering COX-2 inhibitors, in many cases the presence of a permeation promoter is worthwhile, such as, for example, fatty alcohols such as decanol, dodecanol, fatty acids such as oleic acid, myristic acid and polyoxyethylene fatty alcohol ethers. Of these, polyoxy lauryl ether (Brij®) is preferably employed. Also suitable are polyoxyethylene fatty acid esters and fatty acid esters such as sorbitan monolaurate or esters of long-chain fatty acids with methyl, ethyl or isopropyl alcohol or esters of fatty alcohols with acetic acid or lactic acid, and substances such as oleic acid diethanolamine.

### EXAMPLES

### Example 1

Production of a TTS

The TTS of the invention can be produced as follows, for example:

50 g of rofecoxib and 20 g of a suitable permeation-promoting substance (e.g. Brij®30) are dissolved in 200 g of 1,2-propanediol. This solution is added, using a suitable stirring apparatus, to the silicone adhesive 4301 from Dow Corning (USA) and dispersed so that, if possible, a homogeneous liquid/liquid dispersion is produced. This dispersion is homogeneously coated, using a simple apparatus, onto a support sheet, e.g. made of polyethylene terephthalate. Subsequent controlled drying removes the solvent of the silicone adhesive and any propanediol contents. The laminate obtained in this way is subsequently laminated onto another sheet of polyethylene terephthalate. Finally, TTS of a particular area are punched out and packed in a suitable packaging.

### Example 2

Production of an STS

The STS of the invention can be produced as follows, for example:

50 g of rofexoxib are dissolved in 200 g of 1,2-propanediol. This solution is added, using a suitable stirring apparatus, to the silicone adhesive 4301 from Dow Corning (USA) and dispersed so that, if possible, a homogeneous liquid/liquid dispersion is produced. This dispersion is homogeneously coated, using a simple apparatus, onto an elastic support fabric, e.g. made of polyethylene terephthalate. Subsequent controlled drying removes the solvent of the silicone adhesive and any propanediol contents. The laminate obtained in this way is subsequently laminated onto another sheet of polyethylene terephthalate. Finally, STS of a particular area are punched out and packed in a suitable packaging.

1. A dermal therapeutic system comprising at least one active pharmaceutical ingredient for the treatment of symptoms associated with irritation from degenerative joint disorders, and for the treatment of acute, postoperative and menstrual pain, characterized in that at least one of the active ingredients is a COX-2 inhibitor.

2. A dermal therapeutic system as claimed in claim 1, characterized in that the active ingredient is a 3,4-diaryl-2(5H)-furanone.

3. A dermal therapeutic system as claimed in claim 2, characterized in that the COX-2 inhibitor is a 3-aryl-4-(4-methylsulfonylphenyl)-2(5H)furanone.

4. A dermal therapeutic system as claimed in claim 3, characterized in that the COX-2 inhibitor is 3-phenyl-4-(4-methylsulfonylphenyl)-2(H5)furanone (rofecoxib).

5. A dermal therapeutic system as claimed in claim 1, characterized in that the COX-2 inhibitor is 4-(5-aryl-3-trifluoromethylpyrazol-1-yl)benzenesulfonamide.

6. A dermal therapeutic system as claimed in claim 5, characterized in that the COX-2 inhibitor is 4-[5-(4-methylphenyl)-3-(trifluoromethyl)pyrazol-1-yl]benzenesulfonamide (celecoxib).

7. A dermal therapeutic system as claimed in claim 1, characterized in that the COX-2 inhibitor is meloxicam.

8. A dermal therapeutic system as claimed in claim 1, characterized in that the COX-2 inhibitor is nimesulide.

9. A dermal therapeutic system as claimed in one or more of claims 1-8, characterized in that it is a transdermal therapeutic system (TTS).

10. A dermal therapeutic system as claimed in one or more of claims 1-8, characterized in that it is a superficial therapeutic system (STS).

11. A dermal therapeutic system as claimed in one or more of the preceding claims, characterized in that the COX-2 inhibitor concentration in the system is in the range from 0.1 to 50% by weight.

12. A dermal therapeutic system as claimed in claim 11, characterized in that the COX-2 inhibitor concentration is from 1 to 10% by weight.
